(19) United States
(12) Patent Application Publication
(10) Pub. No.: US 2010/0311766 A1
(43) Pub. Date: Dec. 9, 2010

(54) ANTIBACTERIAL COMPOSITIONS

(75) Inventors: David John Haydon, Oxfordshire (GB); Lloyd George Czaplewski, Oxfordshire (GB)

Correspondence Address: BANNER & WITCOFF, LTD., 1100 13th STREET, N.W., SUITE 1200, WASHINGTON, DC, 20005-4051, US

(73) Assignee: BIOTA EUROPE LTD, Oxfordshire (GB)

(21) Appl. No.: 12/747,753

(22) PCT Filed: Dec. 12, 2008
(86) PCT No.: PCT/GB08/04110

§ 371 Date: Jul. 14, 2010

(30) Foreign Application Priority Data
Dec. 13, 2007 (GB) 0724349.6

(51) Int. Cl. A61K 31/497; C07D 513/04; A61K 31/444; A61P 31/04; A01N 43/40; A01N 43/60; A01P 1/00

(52) U.S. Cl. 514/255.05; 546/114; 544/405; 514/301

(57) ABSTRACT

Compounds of formula (I) have antibacterial activity wherein: m is O or 1; Q is hydrogen or cyclopropyl; AIk is an optionally substituted, divalent C₁-C₃ alkylene, C₂-C₃ alkenylene or C₂-C₃ alkynylene radical; X is —C(═O)NH— or —C(═O)O—; R₂ and R₃ are as defined in the specification.

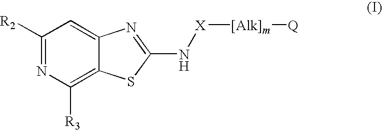

This invention relates to substituted thiazolopyridines that are useful as antibacterial agents.

### BACKGROUND TO THE INVENTION

Type II topoisomerases catalyse the interconversion of DNA topoisomers by transporting one DNA segment through another. Bacteria encode two type II topoisomerase enzymes, DNA gyrase and DNA topoisomerase IV. Gyrase controls DNA supercoiling and relieves topological stress. Topoisomerase IV decatenates daughter chromosomes following replication and can also relax supercoiled DNA. Bacterial type II topoisomerases form a heterotetrameric complex composed of two subunits. Gyrase forms an A₂B₂ complex comprised of GyrA and GyrB whereas topoisomerase forms a C₂E₂ complex comprised of ParC and ParE. In contrast eukaryotic type II topoisomerases are homodimers. Ideally, an antibiotic based on the inhibition of bacterial type II topoisomerases would be selective for the bacterial enzymes and be relatively inactive against the eukaryotic type II isomerases. The type II topoisomerases are highly conserved enzymes allowing the design of broad-spectrum inhibitors. Furthermore, the GyrB and ParE subunits are functionally similar, having an ATPase domain in the N-terminal domain and a C-terminal domain that interacts with the other subunit (GyrA and ParC respectively) and the DNA. The conservation between the gyrase and topoisomerase IV active sites suggests that inhibitors of the sites might simultaneously target both type II topoisomerases. Such dual-targeting inhibitors are attractive because they have the potential to reduce the development of target-based resistance.

Type II topoisomerases are the target of a number of antibacterial agents. The most prominent of these agents are the quinolones. The original quinolone antibiotics included nalidixic acid, cinoxacin and oxolinic acid. The addition of fluorine yielded a new class of drugs, the fluoroquinolones, which have a broader antimicrobial spectrum and improved pharmacokinetic properties. The fluoroquinolones include norfloxacin, ciprofloxacin, and fourth generation quinolones gatifloxacin and moxifloxacin. The coumarins and the cyclothialidines are further classes of antibiotics that inhibit type II topoisomerases, however they are not widely used because of poor permeability in bacteria, eukaryotic toxicity, and low water solubility. Examples of such antibiotics include novobiocin and coumermycin A1, cyclothialidine, cinodine, and clerocidin. However, the continuous emergence of antibiotic resistance demands that novel classes of antibiotics continue to be developed and alternative compounds that inhibit bacterial topoisomerases are required.

### BRIEF SUMMARY OF THE CONTEXT OF THE INVENTION

This invention is based on the finding that a class of substituted benzothiazoles, thiazolopyridines and thiazolopyridazines has antibacterial activity, as evidenced by inhibition of bacterial growth by members of that class. The compounds exhibit activity against strains of Gram-positive and/or Gram-negative classes, such as staphylococci, enterococci, streptococci, propionibacteria and moraxellas for example Staphylococcus aureus, Staphylococcus epidermidis, Enterococcus faecalis, Enterococcus faecium, Streptococcus pneumoniae, Streptococcus pyogenes, Propionibacterium acnes, Haemophilus influenzae and Moraxella catarrhalis. The compounds with which the invention is concerned are therefore useful for the treatment of bacterial infection or contamination, for example in the treatment of, inter alia, Gram-positive infections, community acquired pneumonias, acne vulgaris, impetigo and infected atopic dermatitis.

Whilst the invention is not limited by any particular hypothesis as to the mechanism of action of the compounds, it is presently believed that such activity is due, at least in part, to the compounds inhibiting the type II bacterial topoisomerases.

The invention therefore encompasses the antibacterial use of the class of substituted thiazolopyridine compounds defined herein, and to novel members of that class of compounds.

International Patent Applications WO 03/105846 and WO 2005/012292 relate to benzimidazole, and pyridoimidazole compounds which inhibit bacterial gyrase activity. Many benzimidazoles and a single 1H-imidazo[4,5-b]pyridine were synthesized, characterized and tested for gyrase and antibacterial activity. Although they were claimed, no 3H-imidazo[4,5-c]pyridine was synthesized. Therefore, no “proof of principle” that 3H-imidazo[4,5-c]pyridines have the asserted gyrase inhibitory and antibacterial activities was provided.

Co-pending patent application PCT/GB2007/002314 describes substituted benzothiazole and thiazolopyridine compounds that inhibit bacterial gyrase activity. Antibacterial activity is demonstrated for the benzothiazoles, however no thiazolopyridines were exemplified.

### DESCRIPTION OF THE INVENTION

According to the invention, there is provided a compound of formula (I), or a salt, or N-oxide thereof, in the preparation of an antibacterial composition:

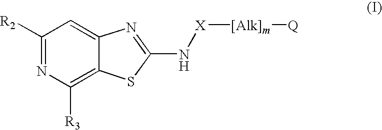

wherein:
m is 0 or 1;
Q is hydrogen or cyclopropyl;
Alk is an optionally substituted, divalent C₁-C₃ alkylene, C₂-C₃ alkenylene or C₂-C₃ alkynylene radical;

### X is —C(═O)NH— or —C(═O)O—;

R₂ is a group Q¹-[Alk¹]_{q}-Q²-, wherein
q is 0 or 1;
Alk¹ is an optionally substituted, divalent, straight chain or branched C₁-C₆ alkylene, or C₂-C₆ alkenylene or C₂-C₆ alkynylene radical which may contain or terminate in an ether (—O—), thioether (—S—) or amino (—NR)— link;
Q² is an optionally substituted divalent monocyclic heterocyclic radical having 5 or 6 ring atoms or an optionally substituted divalent bicyclic heterocyclic radical having 9 or 10 ring atoms;
Q¹ is hydrogen, an optional substituent, or an optionally substituted heterocyclic radical having 3-7 ring atoms;
R is hydrogen, —CN or C₁-C₃ alkyl;
   R₃ is a group Q⁴-[Alk²]ₚ-Q³- other than hydrogen wherein
p is 0 or 1;
Alk² is optionally substituted divalent C₁-C₆ alkylene or C₂-C₆ alkenylene or C₂-C₆ alkynylene radical;
Q³ is an optionally substituted divalent monocyclic heterocyclic radical having 5 or 6 ring atoms or an optionally substituted divalent bicyclic heterocyclic radical having 9 or 10 ring atoms;
Q⁴ is hydrogen, an optional substituent, or optionally substituted heterocyclic ring having 3-7 ring atoms.

In other broad aspects, the invention includes:

(i) the use of a compound (I) as defined above in the preparation of an antibacterial composition;
(ii) a method of treatment of a subject suffering a bacterial infection, or preventing bacterial infection in a subject, comprising administering to the subject an amount of a compound (I) as defined above, sufficient to inhibit bacterial growth;
(iii) a method treating or preventing bacterial contamination of a substrate comprising applying to the site of such contamination or potential contamination an amount of a compound (I) as defined above, sufficient to inhibit bacterial growth;
(iv) a compound (I) as defined above for use in a method of treatment of the human body;
(v) a compound (I) as defined above for use in treating or preventing bacterial infection.

### Terminology

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “divalent (Cₐ-C_{b})alkylene radical” wherein a and b are integers refers to a saturated hydrocarbon chain having from a to b carbon atoms and two unsatisfied valences. The term includes, for example, methylene, ethylene, n-propylene and n-butylene.

As used herein the term “(Cₐ-C_{b})alkenyl” wherein a and b are integers refers to a straight or branched chain alkenyl moiety having from a to b carbon atoms having at least one double bond of either E or Z stereochemistry where applicable. The term includes, for example, vinyl, allyl, 1- and 2-butenyl and 2-methyl-2-propenyl.

As used herein the term “divalent (Cₐ-C_{b})alkenylene radical” means a hydrocarbon chain having from a to b carbon atoms, at least one double bond, and two unsatisfied valences. The term includes, for example, —CH═CH— (vinylene), —CH═CH—CH₂—, —CH₂—CH═CH—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, and —CH═CH—CH₂—CH₂—CH═CH—.

As used herein the term “Cₐ-C_{b} alkynyl” wherein a and b are integers refers to straight chain or branched chain hydrocarbon groups having from a to b carbon atoms and having in addition at least one triple bond. This term would include for example, ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl.

As used herein the term “divalent (Cₐ-C_{b})alkynylene radical” wherein a and b are integers refers to a divalent hydrocarbon chain having from a to b carbon atoms, and at least one triple bond. The term includes, for example, —C≡C—, —C≡C—CH₂—, and —CH₂—C≡CH—.

As used herein the term “carbocyclic” refers to a mono-, bi- or tricyclic radical having up to 16 ring atoms, all of which are carbon, and includes aryl and cycloalkyl.

As used herein the term “cycloalkyl” refers to a monocyclic saturated carbocyclic radical having from 3-8 carbon atoms and includes, for example, cyclopropyl, cyclobutyl, cyclopentyl, cyclohexyl, cycloheptyl. cyclooctyl and bicyclo[2.2.1]hept-1-yl.

As used herein the unqualified term “aryl” refers to a mono-, bi- or tri-cyclic carbocyclic aromatic radical, and includes radicals having two monocyclic carbocyclic aromatic rings which are directly linked by a covalent bond. Illustrative of such radicals are phenyl, biphenyl and naphthyl.

As used herein the unqualified term “heteroaryl” refers to a mono-, bi- or tri-cyclic aromatic radical containing one or more heteroatoms selected from S, N and O, and includes radicals having two such monocyclic rings, or one such monocyclic ring and one monocyclic aryl ring, which are directly linked by a covalent bond. Illustrative of such radicals are thienyl, benzothienyl, furyl, benzfuryl, pyrrolyl, imidazolyl, benzimidazolyl, thiazolyl, benzothiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein the unqualified term “heterocyclyl” or “heterocyclic” includes “heteroaryl” as defined above, and in its non-aromatic meaning relates to a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are azetidinyl, pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

Unless otherwise specified in the context in which it occurs, the term “substituted” as applied to any moiety herein means substituted with up to four compatible substituents, each of which independently may be, for example, (C₁-C₆)alkyl, (C₂-C₆)alkenyl, (C₂-C₆)alkynyl, (C₁-C₆)alkoxy, hydroxy, hydroxy(C₁-C₆)alkyl, (C₁-C₃)alkoxy(C₁-C₃)alkyl, mercapto, mercapto(C₁-C₆)alkyl, (C₁-C₆)alkylthio, halo (including fluoro, bromo and chloro), fully or partially fluorinated (C₁-C₃)alkyl, (C₁-C₃)alkoxy or (C₁-C₃)alkylthio such as trifluoromethyl, trifluoromethoxy, and trifluoromethylthio, nitro, nitrile (—CN), oxo (═O), phenyl, phenyl(C₁-C₃)alkyl-, phenoxy, monocyclic heteroaryl, heteroaryl(C₁-C₃)alkyl-, or heteroaryloxy with 5 or 6 ring atoms, cycloalkyl having 3 to 6 ring carbon atoms, —COOR^{A}, —COR^{A}, —OCOR^{A}, —SO₂R^{A}, —CONR^{A}R^{B}, —CONHNH₂, —SO₂NR^{A}R^{B}, —NR^{A}R^{B}, —NHNH₂, —OCONR^{A}R^{B}, —NR^{B}COR^{A}, —NR^{B}COOR^{A}, —NR^{B}SO₂OR^{A} or —NR^{A}CONR^{A}R^{B} wherein R^{A} and R^{B} are independently hydrogen or a (C₁-C₆)alkyl, hydroxy(C₁-C₆)alkyl, or (C₁-C₃)alkoxy(C₁-C₃)alkyl group or, in the case where R^{A} and R^{B} are linked to the same N atom, R^{A} and R^{B} taken together with that nitrogen may form a cyclic amino ring such as morpholinyl, piperidinyl. piperazinyl, or 4-(C₁-C₆)alkyl-piperizinyl such as 4-methyl-piperazinyl. Where the substituent is phenyl, phenyl(C₁-C₃)alkyl-, phenoxy or monocyclic heteroaryl, heteroaryl(C₁-C₃)alkyl-, or heteroaryloxy with 5 or 6 ring atoms, the phenyl or heteroaryl ring thereof may itself be substituted by any of the above substituents except phenyl, phenyl(C₁-C₃)alkyl-, phenoxy, heteroaryl, heteroaryl(C₁-C₃)alkyl-, or heteroaryloxy. An “optional substituent” or “substituent” may be one of the foregoing specified groups.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds (I) which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesunfonic, glutamic, lactic, and mandelic acids and the like. For a review on suitable salts, see Handbook of Pharmaceutical Salts: Properties, Selection, and Use by Stahl and Wermuth (Wiley-VCH, Weinheim, Germany, 2002).

Compounds of the invention may be prepared in crystalline form, and may be in the form of hydrates and solvates. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water. Reference herein to a compound of the invention is to be understood as including hydrates and solvates thereof.

Compounds with which the invention is concerned which may exist in one or more stereoisomeric form, because of the presence of asymmetric atoms or rotational restrictions, can exist as a number of stereoisomers with R or S stereochemistry at each chiral centre or as atropisomeres with R or S stereochemistry at each chiral axis. The invention includes all such enantiomers and diastereoisomers and mixtures thereof.

Some compounds of formula (I) may be administered as prodrugs, which are considered to be derivatives of compounds of formula (I) which may have little or no pharmacological activity themselves but which, when administered into or onto the body, are converted into compounds of formula (I) having the desired activity, for example, by hydrolytic cleavage. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs can, for example, be produced by replacing appropriate functionalities present in the compounds of formula (I) with certain moieties known to those skilled in the art as ‘pro-moieties’ as described, for example, in Design of Prodrugs by H. Bundgaard (Elsevier, 1985).

Metabolites of compounds of formula (I), that is, compounds formed in vivo upon administration of the drug may also have antibacterial activity. Some examples of metabolites include:

(i) where the compound of formula (I) contains a methyl group, an hydroxymethyl derivative thereof (—CH₃—>—CH₂OH):
(ii) where the compound of formula (I) contains an alkoxy group, an hydroxy derivative thereof (—OR—>—OH);
(iii) where the compound of formula (I) contains a tertiary amino group, a secondary amino derivative thereof (—NR¹R²—>—NHR¹ or —NHR²);
(iv) where the compound of formula (I) contains a secondary amino group, a primary derivative thereof (—NHR¹—>—NH₂);
(v) where the compound of formula (I) contains a phenyl moiety, a phenol derivative thereof (-Ph->-PhOH); and
(vi) where the compound of formula (I) contains an amide group, a carboxylic acid derivative thereof (—CONH₂—>COOH).

### Structural Features

The compounds with which the invention is concerned may have, for example, the following features, in any compatible combination:

m may be 0 and Q may be hydrogen or cyclopropyl.
m may be 1 and Q hydrogen, with Alk being, for example —CH₂—,
—(CH₂)₂— or —(CH₂)₃—. Presently, when m is 1 it is preferred that X be —C(O)NH—, Alk be —(CH₂)₂— and Q be hydrogen.
R₃ is a group Q⁴-[Alk²]-Q³- other than hydrogen.
Alk² when present (ie p is 1) is an optionally substituted divalent C₁-C₆ alkylene or C₂-C₆ alkenylene or C₂-C₆ alkynylene radical, for example optionally substituted —CH₂—, —CH(OH)—, —CH₂CH₂—, —CH₂CH₂CH₂—, —CH═CH—, —C≡C—, —CH₂CH═CH—, —CH₂C≡C—. Presently preferred are optionally substituted divalent C₁-C₃ alkylene radicals

Q³ is an optionally substituted heterocyclic radical having 5 or 6 ring atoms, or an optionally substituted divalent bicyclic heterocyclic radical having 9 or 10 ring atoms. Examples of such radicals include those having optionally substituted thienyl, benzothienyl, furyl, benzfuryl, pyrrolyl, imidazolyl, benzimidazolyl, thiazolyl, benzothiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl, indazolyl. azetidinyl, pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, piperidinyl, piperazinyl, indolyl, morpholinyl, benzfuranyl, pyranyl, isoxazolyl, and benzimidazolyl, rings.

Q⁴ is hydrogen, an optional substituent, or optionally substituted heterocyclic ring having 3-7 ring atoms. Optional substituents include those particularised above in the discussion of the term “optional substituent”. Heterocyclic rings having 3-7 ring atoms include those monocyclic rings listed in the preceding paragraph, as well as cyclopentyl and homopiperazinyl rings.

Currently it is preferred that Q³ be an optionally substituted pyridine ring, an optionally substituted pyrimidine ring or an optionally substituted pyrazine ring, such as an optionally substituted pyridine-2-yl ring, an optionally substituted pyrimidine-2-yl ring or an optionally substituted pyrazine-2-yl ring. Optional substituents in Q³ include CH₃O—, —NH₂, —CN, Cl, CH₃—, and —CF₃.

Examples of radicals R₃ include the following:

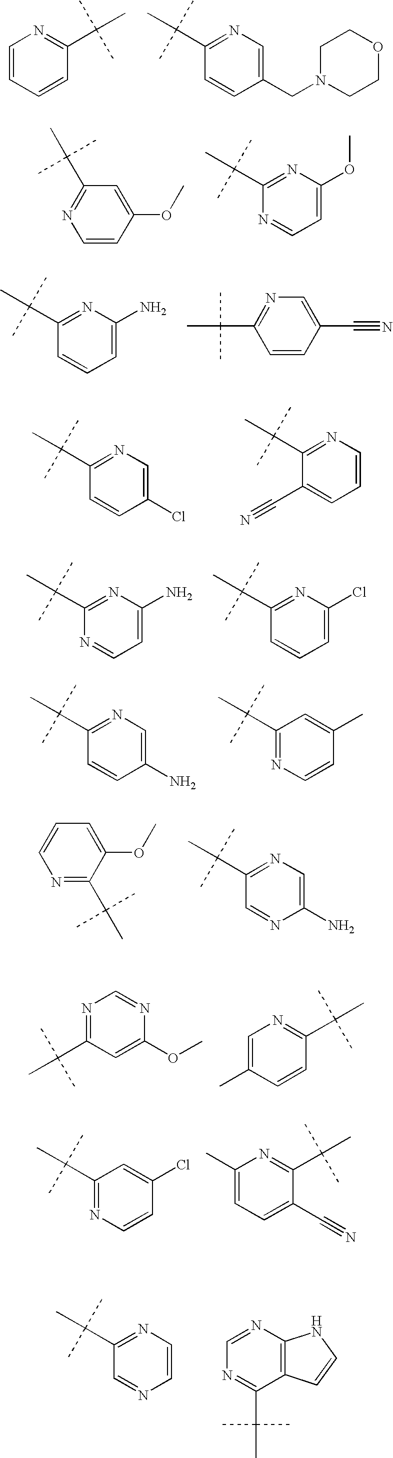

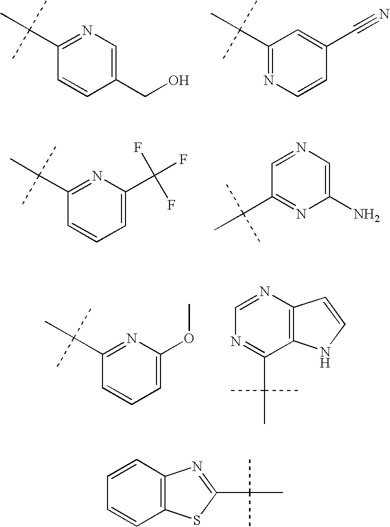

R₂ is a group Q¹-[Alk¹]-Q²-.

Alk¹ when present is an optionally substituted, divalent, straight chain or branched C₁-C₆ alkylene, or C₂-C₆ alkenylene or C₂-C₆ alkynylene radical which may contain or terminate in an ether (—O—), thioether (—S—) or amino (—NR)— link. Examples of such radicals include —CH₂—, —CH(OH)—, —CH₂CH₂—, —CH₂CH₂CH₂—, —CH═CH—, —C≡C—, —CH₂CH═CH—, —CH₂C≡C—, —CH₂NH—, —C(═O)NH—, —CH₂OCH₂—, —CH₂CH₂C(═O)NH—.

Q² is an optionally substituted divalent monocyclic heterocyclic radical having 5 or 6 ring atoms or an optionally substituted divalent bicyclic heterocyclic radical having 9 or 10 ring atoms. Examples of such radicals include those specified above in the discussion of radical Q³.

Q¹ is hydrogen, an optional substituent, or an optionally substituted heterocyclic radical having 3-7 ring atoms. Examples of such radicals include those specified above in the discussion of radical Q⁴.

In the group R₂, Q² may be an optionally substituted divalent nitrogen-containing heterocyclic radical having 5 or 6 ring atoms, such as an optionally substituted divalent pyridonyl, pyridyl, pyrazolyl, pyrimidinyl, thiazolyl, or pyrrolyl radical, or Q₂ when present may be a divalent nitrogen-containing bicyclic or heterocyclic radical having 9 or 10 ring atoms, such as quinolinyl, isoquinolinyl, benzimidazolyl or 5-azaindolyl. Presently preferred Q² rings include optionally substituted pyridine, pyrimidine, pyrazine or pyridine-2-one rings, such as an optionally substituted pyridine-3-yl ring, an optionally substituted pyrimidine-5-yl ring, an optionally substituted pyrazine-2-yl ring or an optionally substituted pyridine-2-one-4-yl ring. Presently preferred optional substituents in Q² include CH₃—, CH₃O—, —CN, and —NH₂.

In the group R₂, q is 0 or 1. When q is 1, Alk¹ is present and may be, for example, an optionally substituted divalent C₁-C₃ alkylene radical which may optionally include an —NH— link, or optionally terminate in an —NH— link to Q². In a particular case, Alk¹ is a divalent C₂-C₃ alkylene radical which terminates in an —NH— link to Q², and which is oxo-substituted on the C atom adjacent that —NH— link, whereby Alk¹ has the formula —(CH₂)₀₋₂C(═O)NH—. In other cases Alk¹ has the formula —(CH₂)₁₋₂NHC(═O)—, with the (C═O) being linked to Q².

In the group R₂, Q¹ may be, for example, hydrogen, or an optional substituent as particularised above. In some embodiments Q¹ is a group of formula —NR^{A}R^{B}, wherein R^{A} and R^{B} are independently hydrogen or a (C₁-C₆)alkyl, hydroxy(C₁-C₆)alkyl, or (C₁-C₃)alkoxy(C₁-C₃)alkyl group, or R^{A} and R^{B} taken together with that nitrogen form a cyclic amino ring, for example, a piperidine, morpholine, thiomorpholine, azetidine, pyrrolidine or piperazine ring, the latter being optionally N-substituted by C₁-C₃ alkyl.

Examples of radicals R² include the following:

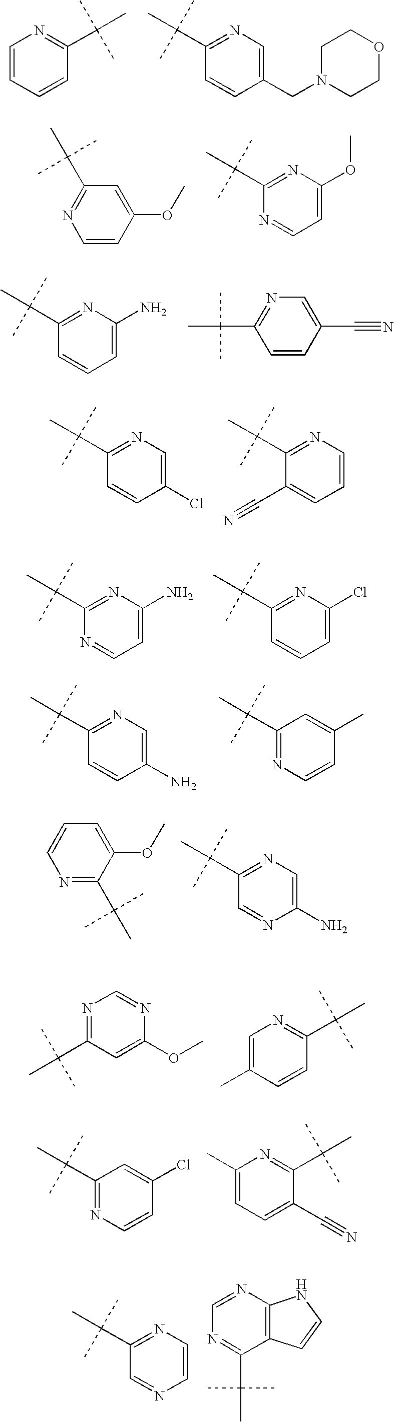

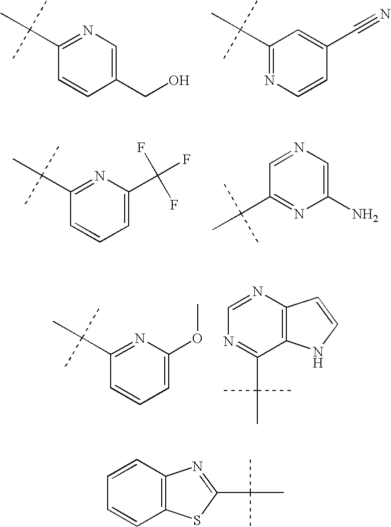

### Utilities and Compositions

As mentioned above, the compounds with which the invention are concerned are antimicrobially active, and may therefore be of use as topical antibacterial disinfectants, or in the treatment of microbial infection in humans and non-human animals e.g. other mammals, birds and fish. Since the type II topoisomerase target of the compounds of the invention is a universal bacterial enzyme, the compounds of the invention inhibit growth of a variety of bacterial species, of the Gram-positive and/or Gram negative classes, such as staphylococci, enterococci, streptococci, and moraxellas for example Staphylococcus aureus, Staphylococcus epidermidis, Enterococcus faecalis, Enterococcus faecium, Streptococcus pneumoniae, Streptococcus pyogenes and Moraxella catarrhalis. The compounds with which the invention is concerned are therefore useful for the treatment of bacterial infection or contamination, for example in the treatment of, inter alia, Gram-positive infections and community acquired pneumonias.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy. Optimum dose levels and frequency of dosing will be determined by clinical trial as is required in the art.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

For topical application to the eye, the drug may be made up into a solution or suspension in a suitable sterile aqueous or non aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The active ingredient may be inhaled using a suitable device such as a dry powder inhaler, a nebuliser, a metered dose inhaler or a liquid spray system.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

### Synthesis and Example Compounds

There are multiple synthetic strategies for the synthesis of the compounds (I) with which the present invention is concerned, but all rely on known chemistry, known to the synthetic organic chemist. Thus, compounds according to formula (I) can be synthesised according to procedures described in the standard literature and are well-known to one skilled in the art. Typical literature sources are “Advanced organic chemistry”, 4^{th} Edition (Wiley), J March, “Comprehensive Organic Transformation”, 2^{nd} Edition (Wiley), R. C. Larock, “Handbook of Heterocyclic Chemistry”, 2^{nd} Edition (Pergamon), A. R. Katritzky), review articles such as found in “Synthesis”, “Acc. Chem. Res.”, “Chem. Rev”, or primary literature sources identified by standard literature searches online or from secondary sources such as “Chemical Abstracts” or “Beilstein”.

Examples of synthetic approaches and schemes for the preparation of compounds (I) are given in the Example herein.

The invention will now be illustrated by reference to the following Example:

### ABBREVIATIONS

DMF—N,N-dimethylformamide
DMSO—dimethylsulfoxide
HPLC—high performance liquid chromatography
MS—mass spectrometry
NMR—nuclear magnetic resonance
Rt—retention time
THF—tetrahydrofuran
TLC—thin layer chromatography

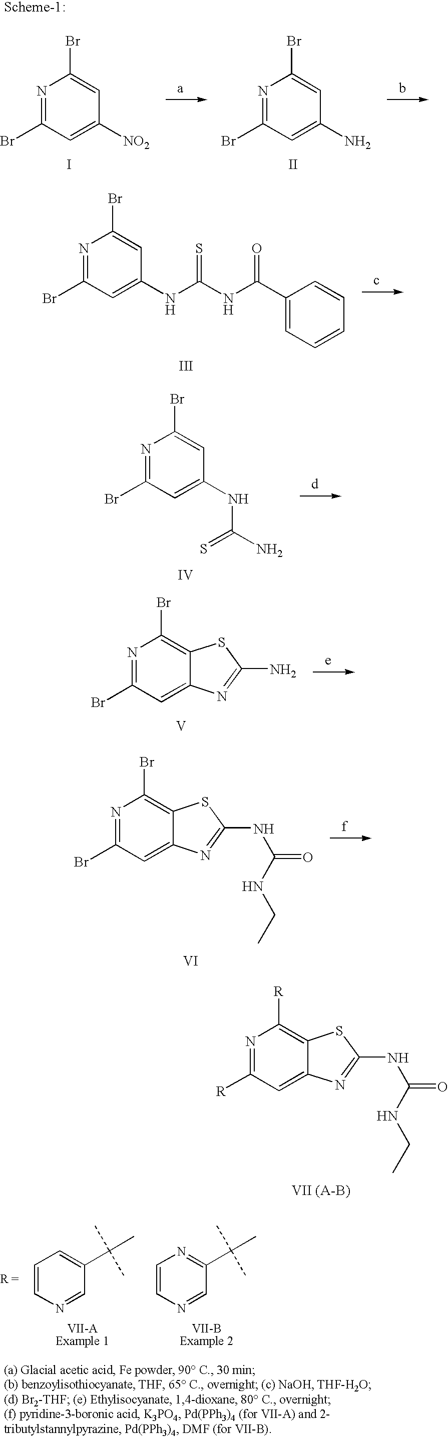

### Preparation of 2,6-Dibromo-pyridin-4-ylamine: II

To a solution of 2,6-dibromo-4-nitro-pyridine (1.0 g, 3.54 mmol) in glacial acetic acid (20 mL) was added Fe-powder (1.0 g, 17.74 mmol) at room temperature. The reaction mixture was refluxed for 90° C. for 30 min. After completion of reaction (TLC monitoring), water was added (100 mL), basified with 2N NaOH (pH 12-14). The resulting mixture was filtered through celite-bed and extracted with ethyl acetate (2×50 mL). The combined organic layers were washed with water, dried (Na₂SO₄), filtered and evaporated to dryness to get the desired product as an off white solid (0.80 g, 90%).

¹H NMR (DMSO-d₆, 400 MHz): δ 6.67 (s, 2H) and 6.71 (br s, 2H).

### Preparation of 1-Benzoyl-3-(2,6-dibromo-pyridin-4-yl)-thiourea: III

To a solution of 2,6-dibromo-pyridin-4-ylamine (0.80 g, 3.17 mmol) in THF (50 mL) was added benzoylisothiocyanate (0.47 mL, 3.49 mmol). The reaction mixture was heated to 65° C. overnight. After completion of reaction (TLC monitoring), THF was distilled off and the crude solid was filtered and washed with hexane (2×50 mL) to get the desired product as a pale-yellow solid (1.20 g, 91%).

¹H NMR (DMSO-d₆, 400 MHz): δ 7.55 (m, 2H), 7.68 (m, 1H), 7.96 (d, J=1.20 Hz, 2H), 8.27 (s, 2H), 11.93 (br s, 1H) and 12.75 (br s, 1H).

### Preparation of (2,6-Dibromo-pyridin-4-yl)-thiourea: IV

To a solution of 1-benzoyl-3-(2,6-dibromo-pyridin-4-yl)-thiourea (1.0 g, 2.14 mmol) in THF (50 mL) was added NaOH solution (0.48 in 20 mL H₂O) at room temperature. The reaction mixture was heated up to 60-65° C. overnight. After completion of reaction (TLC monitoring) THF was distilled off followed by addition of water, and extraction with ethyl acetate (2×50 mL). The combined organic layers were dried over anhydrous Na₂SO₄, filtered and evaporated to dryness under reduced pressure. The crude residue was washed with mixture of ethyl acetate:hexane (30:70) and dried under high vacuum to get the desired product as a white solid (0.58 g, 78%).

¹H NMR (DMSO-d₆, 400 MHz): δ 7.75 (br s, 1H), 7.97 (s, 2H), 8.64 (br s, 1H) and 10.35 (br s, 1H). MS: 309.9 (M+H⁺).

### Preparation of 4,6-Dibromo-thiazolo[5,4-c]pyridin-2-ylamine: V

To a cooled (−60-65° C.) solution of 2,6-dibromo-pyridin-4-yl-thiourea (0.50 g, 1.61 mmol) in THF (100 mL) was added bromine solution (0.20 mL in 45 mL THF, 3.69 mmol) drop wise over a period of 30 min maintaining the temperature to −60-65° C. The reaction mixture was stirred for 15 min at the same temperature and then slowly allowed to come to room temperature. The resulting mixture was heated up to 40° C. for 5 h. After completion of reaction (TLC monitoring), THF was distilled off, basified with aq. NH₃ (25% solution, pH 10-12) and then extracted with ethyl acetate (3×50 mL). The combined organics was washed with water, dried (Na₂SO₄), filtered and concentrated. The crude residue was purified over silica gel (100-200 M, 10% ethyl acetate:hexane) to get the desired product as a white solid (0.10 g, 20%).

¹H NMR (DMSO-d₆, 400 MHz): δ 7.52 (s, 1H) and 8.60 (s, 2H).

### Preparation of 1-(4,6-Dibromo-thiazolo[5,4-c]pyridin-2-yl)-3-ethyl-urea: VI

To a solution of 4,6-dibromo-thiazolo[5,4-c]pyridin-2-ylamine (0.10 g, 0.33 mmol) in 1,4-dioxane (15 mL) was added ethylisocyanate (0.15 mL, 1.94 mmol) and the reaction mixture was heated up to 78-80° C. overnight. After completion of reaction (TLC monitoring) 1,4-dioxane was distilled off and co evaporated with hexane. The solid residue was treated with water to 60-70° C. for 3-5 h. The resulting solid was filtered off and again washed with hot water, dried under high vacuum, purified through column chromatography (ethyl acetate:hexane, 15:85) to obtain the desired product as a pale-yellow solid (0.066 g, 54%).

¹H NMR (DMSO-d₆, 400 MHz): δ 1.09 (t, J=7.20 Hz, 3H), 3.20 (m, 2H), 6.87 (br s, 1H), 7.88 (s, 1H) and 11.55 (br s, 1H).

### Preparation of 1-(4,6-Di-pyridin-3-yl-thiazolo[5,4-c]pyridin-2-O-3-ethyl-urea: VII-A (Example 1)

To a solution of 1-(4,6-dibromo-thiazolo[5,4-c]pyridin-2-yl)-3-ethyl-urea (0.07 g, 0.17 mmol) in DMF:H₂O (3:1, 4 mL) was added pyridine-3-boronic acid (0.08 g, 0.68 mmol) and potassium phosphate tribasic (0.09 g, 0.43 mmol) at room temperature under nitrogen atmosphere. The reaction mixture was degassed by bubbling nitrogen for 15-20 min followed by addition of Pd(PPh₃)₄ (0.04 g, 0.07 mmol). The resulting solution was again degassed for 15-20 min followed by heating up to 90° C. overnight. After completion of reaction (TLC monitoring), the reaction mixture was cooled, water was added and extracted with ethyl acetate, dried over Na₂SO₄ and evaporated to dryness under high vacuum. The crude residue was purified by Prep HPLC to get the desired product (0.015 g, 23%).

¹H NMR (DMSO-d₆, 400 MHz): δ 1.10 (t, J=7.20 Hz, 3H), 3.20 (m, 2H), 6.90 (br s, 1H), 7.54 (m, 1H), 7.68 (m, 1H), 8.31 (s, 1H), 8.49 (m, 1H), 8.61 (m, 2H), 8.74 (m, 1H), 9.30 (d, J=2.0 Hz, 1H), 9.44 (d, J=1.6 Hz, 1H) and 11.46 (br s, 1H). MS: 377.11 (M+H⁺).

Qualitative HPLC Purity (Acquity BEH C-18, 2.1×100 mm, 1.7 μm): 99.24% (Rt=3.05 min).

### Preparation of 1-(4,6-di(pyrazin-2-yl)thiazolo[5,4-c]pyridin-2-yl)-3-ethylurea: VII-B (Example 2)

To a solution of 1-(4,6-dibromo-thiazolo[5,4-c]pyridin-2-yl)-3-ethyl-urea (0.06 g, 0.16 mmol) in DMF (3 mL) was added 2-(tributylstannyl)pyrazine (0.10 mL, 0.32 mmol) at room temperature under nitrogen atmosphere. The reaction mixture was degassed by bubbling nitrogen for 10-15 min followed by addition of Pd(PPh₃)₄ (0.018 g, 0.015 mmol). The resulting solution was again degassed for 15-20 min followed by heating up to 95° C. overnight. After completion of reaction (TLC monitoring), the reaction mixture was cooled, added water (30 mL) and extracted with ethyl acetate (3×20 mL). The combined organics was dried over Na₂SO₄ and evaporated to dryness under high vacuum. The crude residue was purified over silica-gel (100-200 M, 2% MeOH-DCM) to get the desired product (0.004 g, 7%).

¹H NMR (DMSO-d₆, 400 MHz-Partially soluble): δ 1.12 (t, J=7.20 Hz, 3H), 3.24 (m, 2H), 6.85 (br s, 1H), 8.56 (s, 1H), 8.77 (d=2.40 Hz, 1H), 8.80 (s, 1H), 8.84 (d, J=2.80 Hz, 1H), 8.94 (s, 1H), 9.94 (s, 1H), 10.10 (s, 1H), and 11.27 (br s, 1H). MS: 378.95 (M+H⁺).

### Biological Data

### Minimum Inhibitory Concentration (MIC) Testing

Compounds of this invention were tested for antimicrobial activity by susceptibility testing in liquid or on solid media. MICs for compounds against each strain were determined by the broth microdilution or agar dilution method according to the guidelines of the Clinical Laboratories and Standards Institute, formerly the National Committee for Clinical Laboratory Standards (Clinical Laboratories and Standards Institute. Methods for Dilution Antimicrobial Susceptibility Tests for Bacteria That Grow Aerobically; Approved Standard—Seventh Edition. Document M7-A7. CLSI, Wayne, Pa., 2006; Clinical Laboratories and Standards Institute. Methods for Antimicrobial Susceptibility Testing of Anaerobic Bacteria; Approved Standard—Sixth Edition. Document M11-A6. CLSI, Wayne, Pa., 2004).

Compounds of the current invention were found to have antimicrobial activity in the MIC assays described above.

### Gyrase ATPase Assay

Gyrase converts ATP into ADP and inorganic phosphate. The released phosphate can be detected by the addition of malachite green solution and measured by monitoring the increase in absorbance at 600 nm.

The ATPase assay is carried out in a buffer containing 4.8 μg/ml Gyrase enzyme (A₂B₂ complex from Escherichia coli), 0.08 μg/ml ssDNA, 35 mM Tris pH 7.5, 24 mM KCl, 2 mM MgCl₂, 6.5% Glycerol, 2 mM DTT, 1.8 mM Spermidine, 0.5 mg/ml BSA, and 5% DMSO solution containing the inhibitor. The reaction is started by adding ATP to a final concentration of 1 mM and allowed to incubate at 30° C. for 60 minutes. The reaction is stopped by adding 200 μl of malachite green solution (0.034% malachite green, 10 mM ammonium molybdate, 1 M HCl, 3.4% ethanol, 0.01% tween 20). Colour is allowed to develop for 5 minutes and the absorbance at 600 nm is measured spectrophotometrically. The IC₅₀ values are determined from the absorbance readings using no compound and no enzyme controls.

All Example compounds above of the current invention were found to inhibit the gyrase ATPase assay described above, with 50% inhibitory concentrations (IC₅₀) of less than 0.75 micro molar.

The Examples inhibited the growth of bacteria. Table 1 shows the MIC value for each Example against Enterococcus faecalis ATCC 29212 in the MIC Assay described above. Examples with activity “C” demonstrate MICs of 2-16 μg/ml. Examples with activity “B” demonstrate MICs of 0.25-1 μg/ml. Examples with activity “A” demonstrate MICs of <0.25 μg/ml.

TABLE 1
MICs against Enterococcus faecalis

| Example | |
|---|---|
| number | Activity |
| 1 | B |
| 2 | B |

Example compounds were also tested for activity against other bacterial species. For example, Table 2 shows the MICs of Example 1 against various bacterial species. Activity “C” demonstrates an MIC of 2-16 μg/ml. Activity “B” demonstrates an MIC of 0.25-1 μg/ml. Activity “A” demonstrates an MIC of <0.25 μg/ml.

TABLE 2
MICs against various bacteria

| Species | Isolate ID | Activity |
|---|---|---|
| Enterococcus faecalis (VRE) | ATCC 51299 | B |
| Enterococcus faecium (VRE) | ATCC 700221 | C |
| Enterococcus faecium (VSE) | ATCC 19434 | C |
| Moraxella catarrhalis | ATCC 25240 | B |
| Staphylococcus aureus | ATCC 29213 | C |
| Staphylococcus epidermidis | ATCC 12228 | B |
| Staphylococcus haemolyticus | ATCC 29970 | B |
| Streptococcus agalactiae | ATCC 13813 | B |
| Streptococcus mutans | ATCC 35668 | B |
| Streptococcus pneumoniae | ATCC 49619 | B |
| Streptococcus pyogenes | ATCC 51339 | C |

1. A compound of formula (I) or a salt or N-oxide thereof: 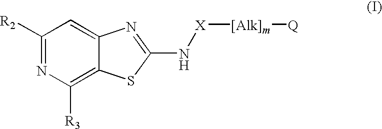
wherein:
m is 0 or 1;
Q is hydrogen or cyclopropyl;
Alk is an optionally substituted, divalent C₁-C₃ alkylene, C₂-C₃ alkenylene or C₂-C₃ alkynylene radical;
X is —C(═O)NH— or —C(═O)O—;
R₂ is a group Q¹-[Alk¹]_{q}-Q²-, wherein
g is 0 or 1;
Alk¹ is an optionally substituted, divalent, straight chain or branched C₁-C₆ alkylene, or C₂-C₆ alkenylene or C₂-C₆ alkynylene radical which may contain or terminate in an ether (—O—), thioether (—S—) or amino (—NR)— link;
Q² is an optionally substituted divalent monocyclic heterocyclic radical having 5 or 6 ring atoms or an optionally substituted divalent bicyclic heterocyclic radical having 9 or 10 ring atoms;
Q¹ is hydrogen, an optional substituent, or an optionally substituted heterocyclic radical having 3-7 ring atoms;
R is hydrogen, —CN or C₁-C₃ alkyl;
R₃ is a group Q⁴-[Alk²]ₚ-Q³- other than hydrogen wherein
p is 0 or 1;
Alk² is optionally substituted divalent C₁-C₆ alkylene or C₂-C₆ alkenylene or C₂-Cₛ alkynylene radical;
Q³ is an optionally substituted divalent monocyclic heterocyclic radical having 5 or 6 ring atoms or an optionally substituted divalent bicyclic heterocyclic radical having 9 or 10 ring atoms;
Q⁴ is hydrogen, an optional substituent, or optionally substituted heterocyclic ring having 3-7 ring atoms.

2. A compound as claimed in claim 1 wherein, in the substituent R₂, Q² is an optionally substituted pyridine, pyrimidine, pyrazine, pyran-2-one or pyridine-2-one ring.

3. A compound as claimed in claim 1 wherein, in the substituent R₃, Q³ is an optionally substituted pyridine ring, an optionally substituted pyrimidine ring or an optionally substituted pyrazine ring.

4. A compound as claimed in claim 1 wherein m is 1 and Q is hydrogen.

5. A compound as claimed in claim 1 wherein X is
—C(O)NH—.

6. A compound as claimed in claim 1 wherein m is 1, Q is hydrogen, Alk is —CH₂CH₂—, and X is —C(O)NH—.

7. A compound as claimed in claim 1 wherein, in the substituent R₂, Q² is an optionally substituted pyridine-3-yl ring, an optionally substituted pyrimidine-5-yl ring, an optionally substituted pyrazine-2-yl ring, an optionally substituted pyran-2-one-4-yl ring or an optionally substituted pyridine-2-one-4-yl ring.

8. A compound as claimed in claim 1 wherein, in the substituent R₂, Alk¹ is present and is an optionally substituted divalent C₁-C₃ alkylene radical

9. A compound as claimed in claim 8 wherein, in the substituent R₂, Q¹ is a group of formula —NR^{A}R^{B}, wherein R^{A} and R^{B} are independently hydrogen or a (C₁-C₆)alkyl, hydroxy(C₁-C₆)alkyl, or (C₁-C₃)alkoxy(C₁-C₃)alkyl group.

10. A compound as claimed in claim 8 wherein, in the substituent R₂, Q¹ is a group of formula —NR^{A}R^{B}, wherein R^{A} and R^{B} taken together with that nitrogen form a cyclic amino ring.

11. A compound as claimed in claim 10 wherein the cyclic amino ring is a morpholinyl, piperidinyl, or piperazinyl ring.

12. A compound as claimed in claim 1 wherein, in the substituent R₃, p is 1.

13. A compound as claimed in claim 12 wherein, in the substituent R₃, Alk² is an optionally substituted divalent C₁-C₃ alkylene radical.

14. A compound as claimed in claim 1 wherein, in the substituent R₃, Q⁴ is hydrogen and p is 0.

15. A compound as claimed in claim 1 wherein, in the substituent R₃, Q³ is an optionally substituted pyridine-2-yl ring, an optionally substituted pyrimidine-2-yl ring or an optionally substituted pyrazine-2-yl ring.

16. A compound as claimed in claim 1 selected from the group consisting of:
1-(4,6-di-pyridin-3-yl-thiazolo[5,4-c]pyridin-2-yl)-3-ethyl-urea
1-(4,6-di(pyrazin-2-yl)thiazolo[5,4-c]pyridin-2-yl)-3-ethylurea.

17. An antibacterial composition comprising a compound as claimed claim 1, together with one or more pharmaceutically acceptable carriers and/or excipients.

18. (canceled)

19. A method of treating or preventing bacterial contamination of a substrate comprising applying to the site of such contamination or potential contamination an amount of a compound (I) as defined in claim 1, sufficient to inhibit bacterial growth

20. A method of treatment of a subject suffering a bacterial infection, or preventing bacterial infection in a subject, comprising administering to said subject an antibacterially effective amount of a compound as defined in claim 1.
